(19) United States
(12) Patent Application Publication
(10) Pub. No.: US 2020/0155118 A1
(43) Pub. Date: May 21, 2020

(54) METHODS AND SYSTEMS FOR AUTOMATIC BEAM STEERING

(71) Applicant: General Electric Company, Schenectady, NY (US)

(72) Inventors: Robert John Anderson, Oshkosh, WI (US); Menachem Halmann, Monona, WI (US); Cynthia Owen, Powhatan, AR (US)

(21) Appl. No.: 16/195,631

(22) Filed: Nov. 19, 2018

(51) Int. Cl. A61B 8/00; A61B 8/08; G06T 7/00; G06T 7/215; A61B 17/34

(52) U.S. Cl. CPC A61B 8/4461; A61B 8/0841; A61B 8/5207; A61B 8/54; A61B 2017/3413; G06T 7/215; A61B 17/3403; G06T 2207/10132; G06T 2207/30004; G06T 7/0014

(57) ABSTRACT

Various methods and systems are provided for guiding maneuvering of a needle with automatic ultrasound beam steering. As one example, the ultrasound beam transmitted from a probe is steered automatically in response to a location of a tissue motion detected from ultrasound images acquired by the probe.

### FIELD

Embodiments of the subject matter disclosed herein relate to medical ultrasound imaging, and more particularly, to automatic beam steering for needle visualization.

### BACKGROUND

Ultrasound is a non-invasive imaging modality that employs ultrasound waves to probe the acoustic properties of an image object (e.g., the body of a patient) and produce a corresponding image. For example, sound wave pulses of a particular frequency range are transmitted into the image object from an ultrasound probe positioned at the surface (such as skin) of the image object. The ultrasound probe may include one or more transducer elements. After transmitting the sound wave pulses, the ultrasound probe may be switched to a receiving mode and collect sound waves scattered back from various depths of the image object. The received sound waves may be constructed into an ultrasound image based on the receiving time. Ultrasound imaging may be used to guide needle maneuver in real-time during procedures such as biopsy and injection.

### BRIEF DESCRIPTION

In one embodiment, a method comprises automatically adjusting ultrasound beam steering angle of a probe in response to a location of a tissue motion in an ultrasound image detected by the probe. In this way, the needle may be visualized in the ultrasound image.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example ultrasound imaging system according to an embodiment of the invention.

FIG. 2 shows an example method for steering an ultrasound beam automatically based on motion in an imaging region.

FIG. 3 shows an example subroutine for selecting a section of the imaging region with the highest motion.

FIG. 4 illustrates beam steering angles when the imaging region contains four quadrants.

FIG. 5 shows an example method for operating an ultrasound system with automatic beam steering.

FIG. 6 illustrates operating the ultrasound imaging system with automatic beam steering before inserting a needle.

FIG. 7 illustrates operating the ultrasound imaging system with automatic beam steering while the needle is within the tissue.

### DETAILED DESCRIPTION

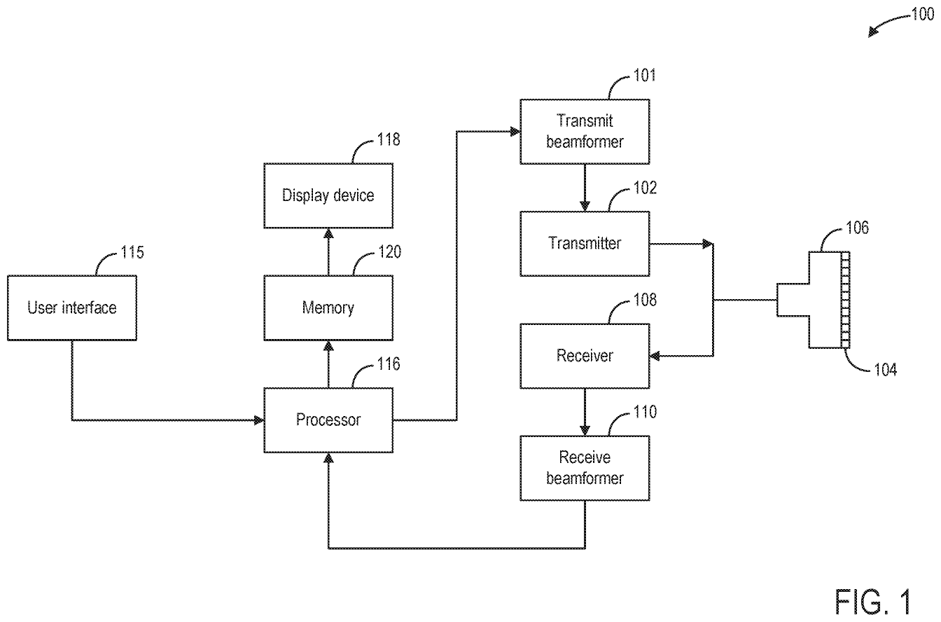

The following description relates to various embodiments of monitoring needle position with an ultrasound imaging system, such as the ultrasound imaging system of FIG. 1. The ultrasound imaging system includes an ultrasound probe for transmitting and receiving sound waves. The transmitted sound waves may form an ultrasound beam directed to an image object. During procedures such as injection or biopsy, the operator (such as a physician) may use ultrasound imaging to guide needle maneuver. Signal strength of the needle in the ultrasound image is affected by the angle between the ultrasound beam and the orientation of the needle. For example, the needle may appear the brightest when the ultrasound beam is perpendicular to the needle orientation; and the needle may be barely visible when the ultrasound beam is parallel to the needle orientation. In order to visualize the needle position, the operator may need to manually steer the ultrasound beam, for example, by pressing a button of the user interface. However, it may be difficult for the operator to steer the ultrasound beam manually while performing a sterilized procedure. Further, manual ultrasound beam steering may require extensive experience or training for the operator to operate the ultrasound system.

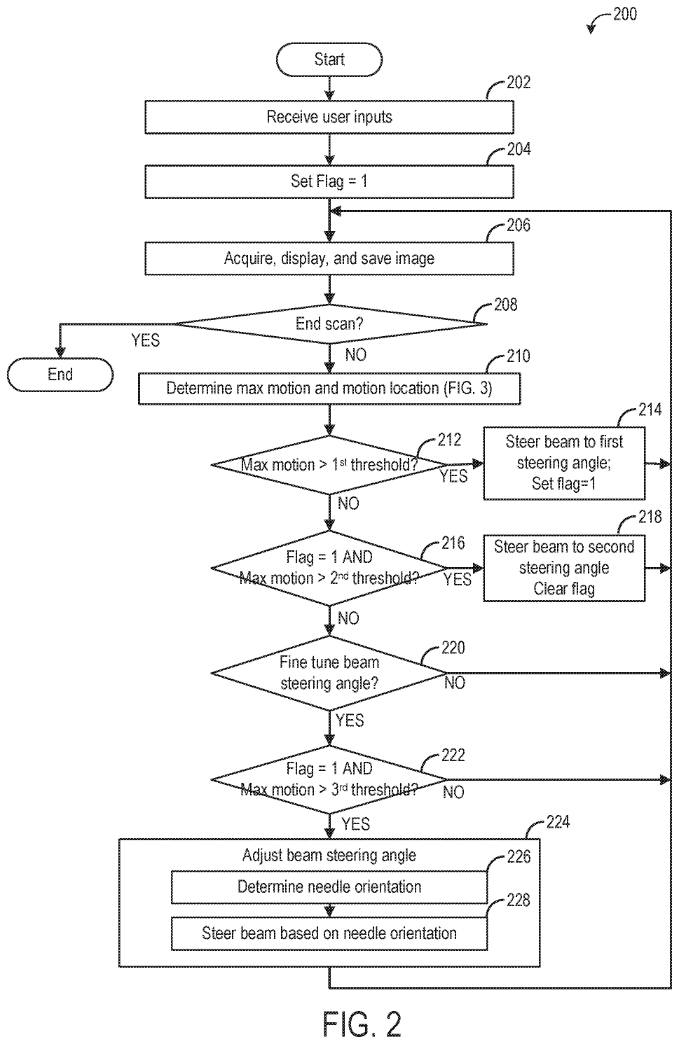

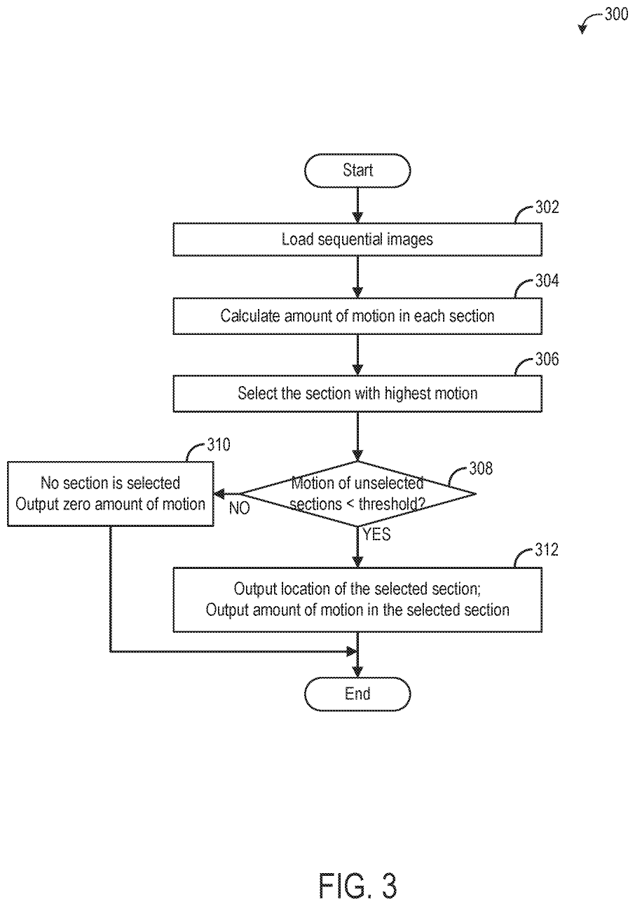

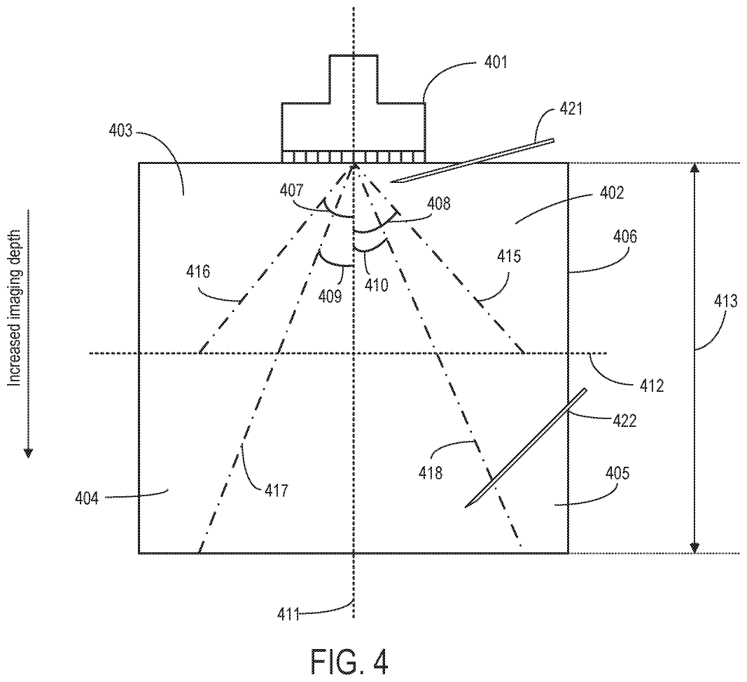

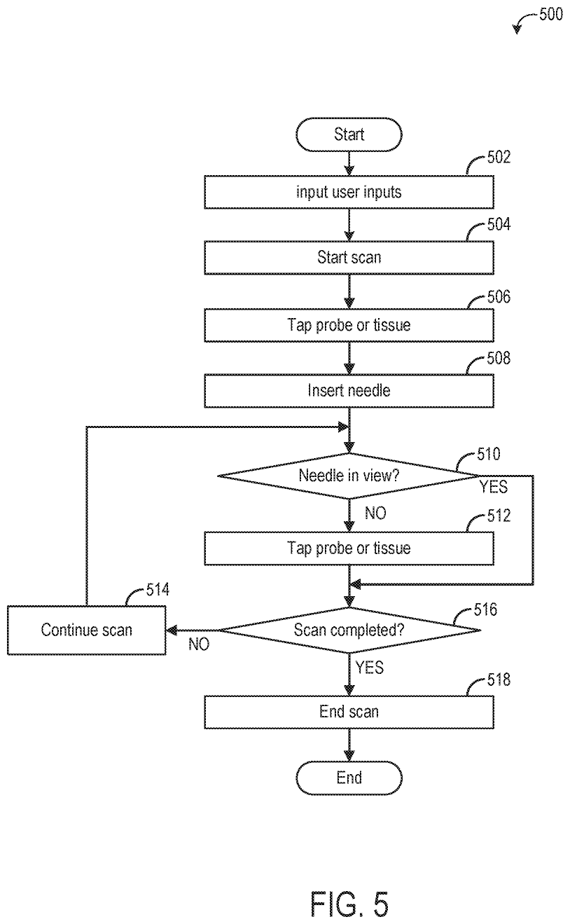

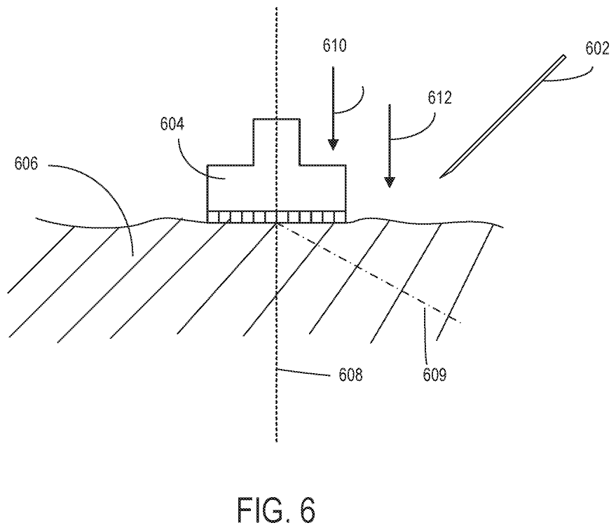

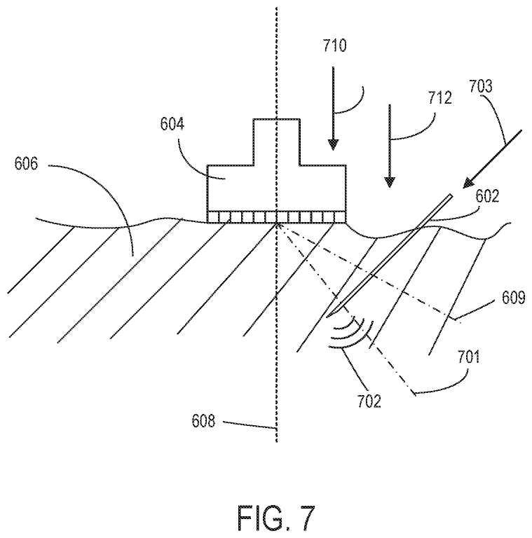

In order to address the above issues, an example method for automatic beam steering is presented in FIG. 2. In particular, an imaging region is scanned by the ultrasound imaging system. The imaging region may be split into multiple predetermined sections. The amount of motion of each section is calculated from sequentially acquired ultrasound images of the imaging region. The ultrasound beam is steered towards a selected section having the maximum amount of motion. FIG. 3 shows an example method of determining the maximum motion. As one example, the imaging region may be split into four quadrants. FIG. 4 illustrates the steering angle for each quadrant when the imaging region is split into four quadrants. FIG. 5 shows an example method for operating the ultrasound imaging system with automatic beam steering shown in FIG. 2 implemented in the system. For example, the operator may trigger automatic beam steering by tapping the ultrasound probe or the skin next to the ultrasound probe, without touching the user interface of the ultrasound system. FIG. 6 and FIG. 7 illustrate operating the ultrasound imaging system with automatic beam steering before and after needle insertion, respectively.

Turning now to FIG. 1, a schematic diagram of an ultrasound imaging system 100 in accordance with an embodiment of the invention is seen. The ultrasound imaging system 100 includes a transmit beamformer 101 and a transmitter 102 that drives elements (e.g., transducer elements) 104 within a transducer array, herein referred to as probe 106, to emit pulsed ultrasonic signals (referred to herein as transmit pulses) into a body (not shown). According to an embodiment, the probe 106 may be a one-dimensional transducer array probe. In some embodiments, the probe 106 may include one transducer element. In some embodiments, the probe 106 may be a two-dimensional matrix transducer array probe. As explained further below, the transducer elements 104 may be comprised of a piezoelectric material. When a voltage is applied to a piezoelectric crystal, the crystal physically expands and contracts, emitting an ultrasonic spherical wave. In this way, transducer elements 104 may convert electronic transmit signals into acoustic transmit beams.

After the elements 104 of the probe 106 emit pulsed ultrasonic signals into a body (of a patient), the pulsed ultrasonic signals are back-scattered from structures within an interior of the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. Additionally, transducer element 104 may produce one or more ultrasonic pulses to form one or more transmit beams in accordance with the received echoes. The elements 104 of the probe may be made of ferroelectric materials, such as piezoelectric ceramic material such as PZT, PMN-PT, PZN-PT, and PIN-PMN-PT single crystal.

The pulsed ultrasonic waves emitted from one or more elements of the probe 106 may form an ultrasound beam. In some embodiments, the ultrasound beam may be focused at a particular depth within the image object by controlling the time of the ultrasound pulse emitted from the transducer elements 104 via the transmit beamformer 101. The ultrasound beam may have a beam path determined based on the location of the pulse emitting elements and the location of the focus. In some embodiments, the beam path of the ultrasound beam may be the central axis of path that the emitted ultrasound pulses propagate within the image object. The ultrasound beam is directed from the probe to the image object along the ultrasound beam path. In one example, the ultrasound beam is directed from the center location of the pulse emitting elements of the probe towards the focus. The ultrasound beam (or the beam path, or the ultrasound beam direction) may be steered from the central axis of the pulse emitting elements by adjusting the timing of each element for emitting the ultrasound pulses.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. A user interface 115 may be used to control operation of the ultrasound imaging system 100, including to control the input of patient data (e.g., patient medical history), to change a scanning or display parameter, to initiate a probe repolarization sequence, and the like. The user interface 115 may include one or more of the following: a rotary element, a mouse, a keyboard, a trackball, hard keys linked to specific actions, soft keys that may be configured to control different functions, and a graphical user interface displayed on a display device 118.

The ultrasound imaging system 100 also includes a processor 116 to control the transmit beamformer 101, the transmitter 102, the receiver 108, and the receive beamformer 110. The processer (or controller) 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on a memory of the processor, and/or memory 120. The processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates raw data. In another embodiment, the demodulation can be carried out earlier in the processing chain. The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay. For example, an embodiment may acquire images at a real-time rate of 7-20 frames/sec. The ultrasound imaging system 100 may acquire 2D data of one or more planes at a significantly faster rate. However, it should be understood that the real-time frame-rate may be dependent on the length of time that it takes to acquire each frame of data for display. Accordingly, when acquiring a relatively large amount of data, the real-time frame-rate may be slower. Thus, some embodiments may have real-time frame-rates that are considerably faster than 20 frames/sec while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on display device 118. Other embodiments may acquire and display data at different rates. For example, some embodiments may acquire data at a frame-rate of less than 10 Hz or greater than 30 Hz depending on the size of the frame and the intended application. A memory 120 is included for storing processed frames of acquired data. In an exemplary embodiment, the memory 120 is of sufficient capacity to store at least several seconds worth of frames of ultrasound data. The frames of data are stored in a manner to facilitate retrieval thereof according to its order or time of acquisition. The memory 120 may comprise any known data storage medium.

In various embodiments of the present invention, data may be processed in different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging) is being performed on a patient. The video processor module may include a separate image memory, and the ultrasound images may be written to the image memory in order to be read and displayed by display device 118.

In various embodiments of the present invention, one or more components of ultrasound imaging system 100 may be included in a portable, handheld ultrasound imaging device. For example, display device 118 and user interface 115 may be integrated into an exterior surface of the handheld ultrasound imaging device, which may further contain processor 116 and memory 120. Probe 106 may comprise a handheld probe in electronic communication with the handheld ultrasound imaging device to collect raw ultrasound data. Transmit beamformer 101, transmitter 102, receiver 108, and receive beamformer 110 may be included in the same or different portions of the ultrasound imaging system 100. For example, transmit beamformer 101, transmitter 102, receiver 108, and receive beamformer 110 may be included in the handheld ultrasound imaging device, the probe, and combinations thereof.

FIG. 2 shows an example method for automatically steering the ultrasound beam responsive to the tissue motion for improved visualization of the needle. The method may be saved as computer readable instructions on a non-transitory memory of the ultrasound imaging system, such as the ultrasound imaging system of FIG. 1. Method 200 may be executed before inserting needle into the imaging region of the image object. The imaging region may be a 2D plane within the image object. The method monitors tissue motion, such as displacement, vibration, and/or combinations thereof, and steers the ultrasound beam towards the location of the tissue motion before and/or during needle insertion. The tissue motion may be caused by external force, such as needle insertion and/or operator tapping the transducer or tissue. The tissue motion may be surface displacement, surface distortion, as well as isolated regions of displacement and vibration and further may include propagating motion waves through the tissue. The ultrasound beam steering angle may be predetermined based on the location of the motion within the imaging region.

At 202, the imaging system receives user inputs. The user inputs may include patient information and the scan protocol. Selecting the scan protocol may include initiate automatic beam steering. The scan protocol may include imaging parameters including imaging depth, field of view, and depth of focus.

At 204, an auto-steering flag is set to one. The automatic ultrasound beam steering is enabled when the auto-steering flag is set (for example, set to one). When the automatic beam steering is enabled, the ultrasound beam may be automatically steered responsive to tissue motion, without the operator's input via the user input. The ultrasound beam steering is disabled when the auto-steering flag is cleared (for example, set to zero). The status of the auto-steering flag may be saved in the memory of the ultrasound imaging system.

At 206, the ultrasound imaging system acquires ultrasound images by transmitting ultrasound beams to the image object, and generates an image of the imaging region based on received signals. The images are sequentially acquired over the same imaging region of the image object at different time points. For example, the images may be continuously acquired at certain frequency (such as 30 frames per second). If the ultrasound beam has not been steered yet, the initial ultrasound beam may be along the central axis of the active elements of the ultrasound probe, that is, elements actively transmitting ultrasound pulses that form the ultrasound beams. In other words, the steering angle is zero. The active elements may be all or a subset of the transducer elements of the probe. In one embodiment, the initial ultrasound beam is aligned with the central axis of the ultrasound probe. The generated images may be stored in the memory, and displayed via the display device (such as display device 118).

At 208, method 200 determines whether the scan is ended. For example, the scan may be ended responsive to the operator's input. In another example, the scan may be ended after the scan duration exceeding a predetermined threshold duration. If the scan ends, method 200 exits. Otherwise, method 200 proceeds to 210.

At 210, the maximum amount of motion and the location of the maximum amount of motion are determined. In one example, the imaging region from which the images are acquired at 206 is split into multiple sections. In another example, the image acquired by the probe at 206 is split into multiple sections. In some embodiments, the imaging region or image may be split into two sections along the central axis of the ultrasound probe. In some embodiments, the imaging region or the image may be split into four quadrants. In some embodiments, the imaging region or the image may be split into more than four sections. The sections may be predetermined when determining the imaging protocol at 202. Each data point in the acquired image at 206 belongs to one of the multiple sections. The amounts of total motion in each of the predetermined sections are calculated, and the section that has the maximum total motion is selected. The ultrasound beam may be steered responsive to the maximum amount of motion of the selected section and the relative location of the selected section within the imaging region.

FIG. 3 shows a subroutine for determining the maximum motion and the location of the maximum motion based on sequentially acquired images at 206.

At 302, sequentially acquired images are loaded, for example, into the processor (such as processor 116). For example, the loaded images may be two most recently acquired images of the imaging region acquired at different time points. In other examples, more than two most recently acquired images may be loaded.

At 304, the amount of motion in each section of the imaging area is calculated. In some embodiments, the amount of motion in a particular section is the total amount of motion determined by all of the data points within the section from sequentially acquired images. For example, the amount of motion in a first section may be determined based on all the data points (or pixels) in the first section of a first image and all the data points (or pixels) in the first section of the second image. The first image and the second image are acquired at different time points. The amount of motion may be calculated by calculating the cross-correlation of all the data points in the first section of the first image and all the data points in the second section of the second image.

At 306, the section with the highest amount of motion is selected. In one example, one section that has the highest amount of motion is selected out of the multiple sections. In another example, if more than one section has the same highest amount of motion, any one of the more than one section may be selected. The rest of the multiple sections in the imaging area are not selected.

At 308, the motion in each of the unselected sections is compared with a threshold motion. For example, the threshold motion may be one fourth of the maximum motion determined at 306. In one embodiment, if the imaging region includes four sections, the motion in each of the three unselected section is compared with the threshold motion. If the motion in each of the unselected section is less than the threshold motion, method proceeds to 312 to output the location of the selected section, and the motion in the selected section. The location of the selected section may be presented by a predetermined index number of the section. For example, the third quadrant may have an index number of three. If the motion in any of the unselected section is greater than the threshold motion, method 300 proceeds to 310, wherein no section is selected, and zero amount of motion is outputted. By not selecting the section and outputting zero amount of motion responsive to the motion in any of the unselected section greater than the threshold motion, tissue motion caused by bulk tissue movement, rather than tissue motion caused by needle insertion or operator tapping, may be eliminated. In this way, the ultrasound beam may be steered based on the local tissue motion (such as the motion in one section) rather than the global motion (such as the motion in the whole imaging region).

In some embodiments, the section with the maximum motion may be selected based on differences between the maximum motion and the motion of each unselected section. For example, the section with the maximum motion may be selected if all of the differences are higher than a threshold motion, and no section is selected if any of the differences is less than the threshold motion.

At 212, the maximum motion determined at 210 is compared with a first threshold motion. For example, the first threshold motion may be 2 mm/sec. In some examples, the first threshold motion may be measured in number of pixels. If the maximum motion is higher than the first threshold motion, method 200 proceeds to 214, wherein the ultrasound beam is steered. Otherwise, method 200 proceeds to 216.

At 214, the ultrasound beam is steered to a first steering angle towards the selected section. The steering angle is the angle between the central axis 411 of the ultrasound probe to the beam path of the steered ultrasound beam. The ultrasound beam is steered within the imaging plane, or the plane of the imaging region. The ultrasound beam is steered to the side of the selected section relative to the central axis of the probe. Further, step 214 sets the auto-steering flag to one.

FIG. 4 illustrates beam steering when the imaging region 406 includes four quadrants. The imaging region 406 includes four quadrants 402, 403, 404, and 405. The central axis of the imaging region 406 is the central axis 411 of the probe 401. The first quadrant 402 and the fourth quadrant 405 are separated from the second quadrant 403 and the third quadrant 404 by the central axis 411 of probe 401. The first quadrant 402 and the second quadrant 403 are separated from the third quadrant 404 and the fourth quadrant 405 by splitting line 412. In one example, the splitting line 412 may be half of the imaging depth 413 of the imaging region. In one embodiment, the area of the first quadrant 402 is the same as the area of the second quadrant 403. The area of the third quadrant 404 is the same as the area of the fourth quadrant 405. In another embodiment, the areas of the four quadrants are the same.

In some embodiments, if it is determined at 210 that the first quadrant 402 or the fourth quadrant 405 has the maximum motion, the ultrasound beam is steered towards the right side of the central axis 411 of the ultrasound probe. Beam path 418 shows an example beam path of the steered ultrasound beam. If it is determined at 210 that the second quadrant 403 or the third quadrant 404 has the maximum motion, the ultrasound beam is steered towards the left side of the central axis 411 of the ultrasound probe. Beam path 417 shows an example beam path of the steered ultrasound beam. The first steering angle may be angle 410 and angle 409. As one example, the first steering angle may be 10 degrees. As another example, the first steering angle may be zero degree, such that the ultrasound beam is not steered. In other examples, the first steering angle may be 20, 30, or 40 degrees.

Turning back to FIG. 2, at 216, if the auto-steering flag is set to one and the maximum motion determined at 210 is greater than the second threshold motion, method 200 proceeds to 218 and steers the ultrasound beam. The second threshold motion may be greater than zero and less than the first threshold motion at 212. Otherwise, method 200 proceeds to 220.

At 218, the ultrasound beam is steered to a second steering angle towards the selected section. The ultrasound beam is steered within the imaging plane, or the plane of the imaging region. The second steering angle may be not greater than the first steering angle. The second steering angle may be determined based on the location of the selected section. In one example, the second steering angle increases with increased depth of the selected section.

In one embodiment, at 218, the auto-steering flag is cleared after steering the beam, so that the ultrasound beam will stay at the second steering angle. In another embodiment, the auto-steering flag is not cleared after steering the beam, so that the ultrasound beam may continue steer to either side of the ultrasound probe.

In some embodiments, the imaging region or image is split into four quadrants as shown in FIG. 4. If the first quadrant 402 is selected, the ultrasound beam may be steered to the beam path 418 with the second steering angle 410. In this way, the steered ultrasound beam may facilitate visualizing needle 421 close to the tissue surface (less imaging depth). In one embodiment, the second steering angle is the same as the first steering angle at step 214. Similarly, if the second quadrant 403 is selected, the ultrasound beam may be steered to the beam path 417 with the second steering angle 409. The second steering angles for the first and second quadrants are the same.

If the selected section is the fourth quadrant 405, the steered ultrasound beam path may be 415, and the second steering angle is 408. The second steering angle may be greater than the second steering angle (such as 409 and 410) for the first and second quadrants. In this way, needle deeper in the tissue (such as needle 422) may be visualized. In one example, the second steering angle is the maximum steering angle achievable by the ultrasound probe. If the selected section is the third quadrant 404, the steered ultrasound beam path is 416, and the second steering angle is 407. The second steering angles for the third and fourth quadrants are the same. As such, the steering angle increases as the depth of the selected section increases in the imaging region. For example, the second steering angle for the first and second quadrants is 10 degrees, and the second steering angle for the third and fourth quadrants is 40 degrees.

As such, the amount of ultrasound beam steering angle may be determined based on the level of the maximum motion determined at 210. For example, responsive to higher motion, the beam is steered to a first, smaller, steering angle. Steered beam directs to the side (such as left or right) of the probe where the maximum motion is detected. In this way, the ultrasound beam may be steered to image needle at shallow imaging depth when high amount of motion is detected. The high amount motion may be caused by external force applied by the operator. For example, the operator may tap the probe or tissue (such as skin surface) near the probe, indicating the needle entrance location. Responsive to lower motion, the beam is steered to a second steering angle. The second steering angle is not smaller than the first steering angle. The lower amount of motion may be caused by the wiggling or the back-and-forth motion of the needle, while the operator maneuvering the needle within the tissue.

In some embodiments, steps 212 and 214 may be omitted, and the ultrasound beam is steered to the second steering angle responsive to the maximum motion higher than the second threshold motion.

At 220, if further adjustment of the beam steering angle is required, method 200 proceeds to 222. Otherwise, method 200 continues acquiring images with the current ultrasound beam.

At 222, if the auto-steering flag is set to one and the maximum motion determined at 210 is higher than the third threshold motion, method 200 proceeds to 224 to fine tune the beam steering angle based on the needle orientation. Otherwise, method 200 continues acquiring images with the current beam steering angle. The third threshold motion is a nonzero level less than the second threshold motion. In one example, the third threshold motion may be one tenth of the second threshold motion. By not steering the ultrasound beam responsive to the low motion (such as motion less than the third threshold), the automatic beam steering is more robust, and is less sensitive to noise.

At 224, the beam steering angle is further adjusted, for example, based on orientation of the needle. In particular, at 226, the orientation of the needle is determined. In some embodiments, the needle orientation may be determined via image segmentation, wherein the needle in the acquired image is identified. In one example, the image segmentation may be performed in the selected section at 210, but not in the other unselected sections. In this way, the image processing time may be reduced. In some embodiments, the needle orientation may be determined based on the direction of tissue motion related to the needle maneuver within the selected section. For example, the needle orientation may be determined based on the characteristic tissue motion caused by the wiggle or back-and-forth maneuvering of the needle.

At 228, the ultrasound beam is steered based on the needle orientation determined at 226. For example, the ultrasound beam may be adjusted closer to a direction perpendicular to the needle orientation to enhance the signal from the needle.

In this way, the ultrasound beam may be automatically steered responsive to tissue motion identified in the selected section of the imaging region for visualizing the needle during the needle procedure. The steering angle for each selected section may be predetermined based on the location of the sections in the imaging region. The motion may be identified via cross-correlation of data in sequentially acquired images. As such, the beam steering angle may be quickly determined in real-time via minimal fast calculations including cross-correlation and thresholding.

In some embodiments, the beam steering angle may further be determined based on the level the motion. This feature enables the operator to steer the ultrasound beam by tapping the ultrasound probe or tissue around the ultrasound probe (as shown in FIG. 5), without direct interaction with the user input of the ultrasound imaging system.

In some embodiments, the automatic beam steering may be disabled or enabled based on the level of the tissue motion. In particular, the automatic beam steering may be disabled after steering the ultrasound beam to the second steering angle, responsive to the amount of motion greater than the second threshold motion and less than the first threshold motion. In this way, frequent beam steering may be avoided. Further, the automatic beam steering may be enabled responsive to the amount of motion higher than the first threshold motion. In this way, the automatic beam steering may be reactivated after being disabled, by tapping the ultrasound probe or tissue around the probe.

FIG. 5 shows an example method 500 for operating an ultrasound system that automatically steers the ultrasound beam according to method 200 of FIG. 2.

At 502, the operator inputs user inputs. The user inputs are the same as the user inputs received at 202 of FIG. 2. The user inputs may include patient information and the scan protocol.

At 504, the operator starts the ultrasound scan. For example, the operator may start the scan by pressing a button of the user interface (such as user interface 115 of FIG. 1).

At 506, the operator may apply an external force to the image object by tapping or pushing one side of the probe or the tissue surface near the side of the probe to steer the ultrasound beam towards the side of the probe that the needle is about to be inserted.

FIG. 6 illustrates auto-steering the ultrasound beam before inserting the needle to one side (such as right) of the ultrasound probe. Before inserting needle 602 into tissue 606 on the right side of the ultrasound probe 604, the operator may tap or briefly push the side of the ultrasound probe relative to the central axis 608 of the ultrasound probe 604, where the needle is going to be inserted. The arrow 610 indicates location and direction for tapping or pushing the ultrasound probe 604. Alternatively, the operator may depress the tissue surface by tapping or briefly pushing the tissue surface on the side of the probe wherein the needled is going to be inserted. The arrow 612 indicates the location and direction for tapping or pushing the tissue 606. The depression induces tissue motion in the imaging region. The tissue motion can trigger the ultrasound system to steer the ultrasound beam towards the side (such as right side in FIG. 6) of the probe that the needle is going to be inserted. As an example, the beam path of the steered beam is shown as 609. In this way, the needle can be visualized as soon as it enters the tissue at 508 of FIG. 5.

At 508, the needle is inserted into the tissue. The ultrasound imaging system may continue monitoring tissue motion and steering the ultrasound beam to a steering angle for improved visualization of the needle.

During the scan, if the operator is not satisfied with the beam steering angle, such as when the needle is not in view at 510, at 512, the operator may tap the side of the probe or tissue surface from where the needle enters the tissue. In this way, the ultrasound beam may be steered to the side of the probe indicated by the operator, without the operator direct interacting with the user interface. If the needle can be visualized at 510, method 500 proceeds to 516.

At 516, if the scan is completed, the operator may end the scan at 518. Otherwise, the operator may continue the ultrasound guided needle maneuver at 514.

FIG. 7 illustrates operating the ultrasound imaging system while inserting the needle 602 into the tissue 606 in the direction 703. The needle 602 is within the tissue 606. The ultrasound imaging system may detect the motion in a predetermined section (such as motion in the fourth quadrant of the imaging region), and steers the ultrasound beam towards the location of the motion. The steered ultrasound beam may be along beam path 701. The detected motion may be induced by the needle 602. For example, the motion may include the back and forth motion along the needle entrance direction 703. The motion may also include motion 702 induced by the operator wigging the needle. In some embodiments, the initial depression at 506 of FIG. 5 may cause the ultrasound beam rotating to the beam path 609. The ultrasound beam may then be automatically rotated to beam path 701 from beam path 609 responsive to the detected motion while the needle is within the tissue. The steering angle of beam path 701 may be smaller than the beam path 609. In some embodiments, the operator may depress the probe or the skin surface near the needle entry point, as shown with 710 and 712, by tapping or pushing the probe or skin, while the needle is within the tissue. The depression may trigger the ultrasound beam steer towards to the side (such as right) of the probe 604, relative to the center axis 608 of the probe. The beam may be steered within the plane of the imaging region.

In this way, the ultrasound guided needle maneuver may be performed with one operator holding the ultrasound probe and the needle. The operator does not need to reach out to the user interface to steer the ultrasound beam. The ultrasound beam may be steered towards the side of needle entering the tissue responsive to tissue movement caused by the operator's tapping of the probe or the tissue.

The technical effect of splitting the imaging region into multiple sections and monitor tissue movement in each section are that the steering angle may be quickly determined with minimal calculation. The technical effect of adjusting the steering angle based on the depth of the motion is that the ultrasound beam may be steered to an angle suitable for visualizing the needle. The technical effect of adjusting the steering angle based on the amount of the movement is that the ultrasound beam may be steered towards the side of the probe which the needle enters the tissue before inserting needle into the tissue.

As one embodiment, a method for medical ultrasound imaging comprises automatically adjusting ultrasound beam steering angle of a probe in response to a location of a tissue motion in an ultrasound image detected by the probe. In a first example of the method, the method further includes wherein the detected motion is induced by needle movement. A second example of the method optionally includes the first example and further includes wherein the detected motion is induced by an external force applied to a skin surface. A third example of the method optionally includes one or more of the first and second examples, and further includes wherein the external force is applied before inserting a needle into a tissue. A fourth example of the method optionally includes one or more of the first through third examples, and further includes, detecting the tissue motion by cross-correlation of the ultrasound image and a previously acquired ultrasound image. A fifth example of the method optionally includes one or more of the first through fourth examples, and further includes, splitting the ultrasound image into a plurality of predetermined sections, wherein the tissue motion is a total amount of motion within each predetermined section of the ultrasound image. A sixth example of the method optionally includes one or more of the first through fifth examples, and further includes, wherein the predetermined sections are two sections separated along a central axis of the ultrasound image. A seventh example of the method optionally includes one or more of the first through sixth examples, and further includes, wherein the predetermined sections are four quadrants. A eighth example of the method optionally includes one or more of the first through seventh examples, and further includes, wherein the location of the tissue motion is the location of the section with the total amount of motion higher than the total amount of motion in any of the other predetermined sections. A ninth example of the method optionally includes one or more of the first through eighth examples, and further includes, wherein adjusting ultrasound beam steering angle of a probe in response to the location of the tissue motion includes steering an ultrasound beam generated by the probe towards the location of tissue motion, the steered ultrasound beam within a plane of the ultrasound image.

As one embodiment, a method for medical ultrasound imaging comprises acquiring images by transmitting an ultrasound beam to an imaging region including a plurality of predetermined sections; determining an amount of motion in each section of the plurality of predetermined sections based on the acquired images; selecting a section with a maximum amount of motion from the plurality of predetermined sections; steering the ultrasound beam with a steering angle determined based on a location of the selected section within the imaging region; and acquiring images with the steered ultrasound beam. In a first example of the method, the method includes not selecting the sections with the amount of motion lower than the maximum amount of motion, and wherein the ultrasound beam is steered responsive to the amount of motion in each and every of the unselected sections lower than a threshold. A second example of the method optionally includes the first example and further includes wherein determining the steering angle based on the location of the selected section includes increasing the steering angle with increased depth of the selected section within the imaging region. A third example of the method optionally includes one or more of the first and second examples, and further includes steering the ultrasound beam to a first steering angle responsive to a first maximum amount of motion higher than a first threshold; and steering the ultrasound beam from the first steering angle to a second, less, steering angle responsive to a second maximum amount of motion lower than the first threshold and higher than a second threshold. A fourth example of the method optionally includes one or more of the first through third examples, and further includes, after steering the ultrasound beam to the second steering angle, not steering the ultrasound beam responsive to a third maximum amount of motion lower than the first threshold and higher than the second threshold. A fifth example of the method optionally includes one or more of the first through fourth examples, and further includes, after steering the ultrasound beam to the second steering angle, steering the ultrasound beam to the first steering angle responsive to a third maximum motion higher than the first threshold. A sixth example of the method optionally includes one or more of the first through fifth examples, and further includes, determining an orientation of a needle within the selected section after steering the ultrasound beam to the second steering angle, and steering the ultrasound beam based on the orientation of the needle.

As one embodiment, an ultrasound imaging system comprises an ultrasound probe; and a controller coupled to the ultrasound probe, the controller with computer readable instructions stored on non-transitory memory that when executed during operation of the ultrasound system, cause the controller to: acquire images of an imaging region including a plurality of predetermined sections with an ultrasound beam along a first ultrasound beam direction; determine a maximum amount of motion in the imaging region based on an amount of motion in each section of the plurality sections; responsive to a maximum amount of motion greater than a first threshold, steer the ultrasound beam from the first ultrasound beam direction to a second ultrasound beam direction; responsive to the maximum amount of motion greater than a second threshold and less than the first threshold, steer the ultrasound beam from the first ultrasound beam direction to a third ultrasound beam direction, wherein a first steering angle between the first ultrasound beam direction and the second ultrasound beam direction is not less than a second steering angle between the first ultrasound beam direction and the third ultrasound beam direction; and acquire images with the steered ultrasound beam. In a first example of the system, the system further includes instructions that when executed, cause the controller to determine the second ultrasound beam direction and the third ultrasound beam direction based on a location of the section with the maximum amount of motion. A second example of the system optionally includes the first example and further includes instructions that when executed, cause the controller to steer the ultrasound beam from the first ultrasound beam direction to the second ultrasound beam direction before inserting a needle to the imaging region.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for medical ultrasound imaging, comprising:
automatically adjusting ultrasound beam steering angle of a probe in response to a location of a tissue motion in an ultrasound image detected by the probe.

2. The method of claim 1, wherein the detected motion is induced by needle movement.

3. The method of claim 1, wherein the detected motion is induced by an external force applied to a skin surface.

4. The method of claim 3, wherein the external force is applied before inserting a needle into a tissue.

5. The method of claim 1, further comprising detecting the tissue motion by cross-correlation of the ultrasound image and a previously acquired ultrasound image.

6. The method of claim 1, further comprising splitting the ultrasound image into a plurality of predetermined sections, wherein the tissue motion is detected based on a total amount of motion within each predetermined section of the ultrasound image compared with one another.

7. The method of claim 6, wherein the predetermined sections are two sections separated along a central axis of the ultrasound image.

8. The method of claim 6, wherein the predetermined sections are four quadrants.

9. The method of claim 6, wherein the location of the tissue motion is the location of the section with the total amount of motion higher than the total amount of motion in each of the other predetermined sections.

10. The method of claim 1, wherein adjusting ultrasound beam steering angle of a probe in response to the location of the tissue motion includes steering an ultrasound beam generated by the probe towards the location of tissue motion, the steered ultrasound beam within a plane of the ultrasound image.

11. A method for medical ultrasound imaging, comprising:
acquiring images by transmitting an ultrasound beam to an imaging region including a plurality of predetermined sections;
determining an amount of motion in each section of the plurality of predetermined sections based on the acquired images;
selecting a section with a maximum amount of motion from the plurality of predetermined sections;
steering the ultrasound beam with a steering angle determined based on a location of the selected section within the imaging region; and
acquiring images with the steered ultrasound beam.

12. The method of claim 11, further comprising not selecting the sections with the amount of motion lower than the maximum amount of motion, and wherein the ultrasound beam is steered responsive to the amount of motion in each and every of the unselected sections lower than a threshold.

13. The method of claim 11, wherein determining the steering angle based on the location of the selected section includes increasing the steering angle with increased depth of the selected section within the imaging region.

14. The method of claim 11, further comprising steering the ultrasound beam to a first steering angle responsive to a first maximum amount of motion higher than a first threshold; and steering the ultrasound beam from the first steering angle to a second, less, steering angle responsive to a second maximum amount of motion lower than the first threshold and higher than a second threshold.

15. The method of claim 14, further comprising after steering the ultrasound beam to the second steering angle, not steering the ultrasound beam responsive to a third maximum amount of motion lower than the first threshold and higher than the second threshold.

16. The method of claim 14, further comprising after steering the ultrasound beam to the second steering angle, steering the ultrasound beam to the first steering angle responsive to a third maximum motion higher than the first threshold.

17. The method of claim 14, further comprising determining an orientation of a needle within the selected section after steering the ultrasound beam to the second steering angle, and steering the ultrasound beam based on the orientation of the needle.

18. An ultrasound imaging system, comprising:
an ultrasound probe; and
a controller coupled to the ultrasound probe, the controller with computer readable instructions stored on non-transitory memory that when executed during operation of the ultrasound system, cause the controller to:
acquire images of an imaging region including a plurality of predetermined sections with an ultrasound beam along a first ultrasound beam direction;
determine a maximum amount of motion in the imaging region based on an amount of motion in each section of the plurality sections;
responsive to a maximum amount of motion greater than a first threshold, steer the ultrasound beam from the first ultrasound beam direction to a second ultrasound beam direction;
responsive to the maximum amount of motion greater than a second threshold and less than the first threshold, steer the ultrasound beam from the first ultrasound beam direction to a third ultrasound beam direction, wherein a first steering angle between the first ultrasound beam direction and the second ultrasound beam direction is not less than a second steering angle between the first ultrasound beam direction and the third ultrasound beam direction; and
acquire images with the steered ultrasound beam.

19. The method of claim 18, further comprising instructions that when executed, cause the controller to determine the second ultrasound beam direction and the third ultrasound beam direction based on a location of the section with the maximum amount of motion.

20. The method of claim 18, further comprising instructions that when executed, cause the controller to steer the ultrasound beam from the first ultrasound beam direction to the second ultrasound beam direction before inserting a needle to the imaging region.
